(19) United States
(12) Patent Application Publication
(10) Pub. No.: US 2023/0285709 A1
(43) Pub. Date: Sep. 14, 2023

(54) NO DELIVERY APPARATUS WITH MANUAL VENTILATION SYSTEM

(71) Applicant: INOSYSTEMS, Antony (FR)

(72) Inventor: Frederic MARCHAL, Antony (FR)

(21) Appl. No.: 18/119,210

(22) Filed: Mar. 8, 2023

(30) Foreign Application Priority Data
Mar. 9, 2022 (FR) 2202056

(51) Int. Cl. A61M 16/12; G05D 7/06; A61M 16/20; A61M 16/00

(52) U.S. Cl. CPC A61M 16/12; A61M 16/0078; A61M 16/202; G05D 7/0652; A61M 2202/0275; A61M 2205/3334; A61M 2205/502

(57) ABSTRACT

Disclosed is an NO supply device including a main gas circuit with NO/N₂ flowrate controller controlled by piloting unit, and a backup circuit including a backup NO circuit and a backup O₂ circuit. The piloting unit are configured in particular to control the NO/N₂ flowrate controller in such a way as to regulate the flowrate of NO/N₂ mixture in the main gas circuit and to direct the gaseous flow obtained in a downstream portion of the backup NO circuit connecting to the backup O₂ circuit in order to form a common backup line including a backup outlet.

This application claims priority to French Application FR 2202056 filed on Mar. 9, 2022, the entire contents of which are hereby incorporated by reference.

### BACKGROUND OF THE INVENTION

### Field of the Invention

The present invention relates to a device or apparatus which supplies gaseous nitric oxide (NO) and which is equipped with a backup system to ensure delivery of NO in normal mode, in backup mode in the event of a fault, or in manual ventilation mode, particularly when passing to manual ventilation by a manual ventilation bag, i.e. a bag valve mask (BVM), when requested by the medical personnel or following a minor defect of the apparatus, and to an installation which serves to administer NO to a patient and comprises such an NO supply device.

### Description of the Related Art

Inhaled nitric oxide, or NOi, is a gaseous medicament commonly used to treat patients suffering from acute pulmonary hypertension, in particular pulmonary vasoconstrictions in adults or children, including the newborn (PPHN), as described for example in EP-A-560928 or EP-A-1516639.

An installation for implementing treatment by NOi, commonly called an NO administration installation, traditionally comprises one or more cylinders containing an NO/N₂ mixture and feeding it to an NO supply device which delivers the NO/N₂ mixture at a controlled flowrate, a respiratory assistance apparatus, also called a medical ventilator, for supplying a respiratory gas containing at least 21% by volume of oxygen, such as an O₂/N₂ mixture or air, to which the NO (i.e. NO/N₂) is added, circuit elements, for example one or more flexible conduits, for conveying the flows of gas between these various items of equipment and to the patient, and a respiratory interface, such as a tracheal probe, for supplying the gaseous mixture containing the NO to the patient. It is also possible to provide a gas humidifier for humidifying the gaseous mixture before its administration to the patient. Such an installation is shown schematically in FIG. 1.

Normally, the NO/N₂ mixture delivered by the NO supply device is injected into the respiratory flow containing at least 21% by volume of oxygen (i.e. air or or an O₂/N₂ mixture) coming from the medical ventilator before being administered to the patient by inhalation in the form of a final respiratory mixture (i.e. an NO/N₂/O₂ or NO/N₂/air mixture) generally containing a few tens of ppmv of NO (ppm by volume) and at least 21% by volume of oxygen O, for example of the order of 1 to 80 ppmv of NO, the remainder being essentially nitrogen (N₂).

Such an NO administration installation is used in a hospital environment to administer the NOi treatment and thus care for patients who need to inhale NO in order to treat their pulmonary hypertension. Examples of such NO administration installations are given in documents WO-A-2012/094008, US-A-2015/320951, US-A-2015/273175, JP-A-H11192303, WO-A-02/40914 and US-A-2003/116159.

In the event of a fault or defect of the NO supply device or of the medical ventilator and/or in the event of its being necessary to ventilate the patient using a manual ventilation bag, when requested by the medical personnel or following a minor defect of the device, the NO supply device must be able to continue supplying the NO/N₂ mixture so that the patient’s treatment is not abruptly interrupted, this being the case for obvious reasons of safety, since an abrupt interruption of the treatment by NOi could be fatal in the case of weakened patients, particularly in the case of neonates suffering from PPHN.

To ensure delivery of NO, even in cases of a fault or in cases of ventilation by manual ventilation bag or in similar situations, it is known to equip the NO supply device with a secondary system or circuit, called the backup circuit, which is entirely pneumatic and designed to deliver an O₂ flowrate that is adjustable between 0 and 20 l/min and a fixed flowrate of NO, for example of the order of 230 ml/min of NO/N₂ mixture, which are mixed with each other in the NO supply device before being injected into a manual ventilation bag. The oxygen is typically supplied from an oxygen source, for example a pressurized oxygen cylinder, which will be connected to the NO supply device. Reference may be made to EP-A-3233171, which teaches the use of such a backup circuit.

The changeover to “backup mode” takes place after the user activates a selection means (i.e. a device) present on the NO supply device, for example a “backup mode” selection button, the activation of which will direct the flows of gas into a secondary circuit, i.e. a backup circuit and/or manual ventilation circuit, of the NO supply device, as is illustrated in FIG. 2.

However, an exclusively pneumatic solution of this kind is not ideal, because the NO concentration of the gaseous mixture obtained (i.e. NO/N₂ + O₂) varies depending on the oxygen flowrate regulated by the user, since the NO flowrate is fixed. Having an NO concentration that varies depending on the chosen flowrate of O₂ is not desirable from the therapeutic point of view, since it must be possible to deliver to the patient a given dosage, that is to say a fixed quantity of NO corresponding to an effective concentration for treating the patient’s pathology, or a reduced dosage in cases where the patient has to be weaned off the ventilator, particularly in manual ventilation mode via a manual insufflator, that is to say a manual ventilation bag or bag valve mask (BVM).

### SUMMARY OF THE INVENTION

In view of the above, the problem addressed is to make available an improved NO supply device which is able to supply a gaseous mixture NO/N₂/O₂ to a patient whether in normal operating mode or in cases of a fault or defect, or during changeover to manual ventilation by a manual ventilation bag or BVM, e.g. when requested by the medical personnel or following a minor defect, preferably while maintaining the desired dosage, that is to say a gaseous mixture NO/N₂/O₂ whose composition is not solely dependent on the oxygen flowrate.

A solution of the invention concerns an NO (i.e. nitric oxide) supply device or apparatus comprising:
piloting means (i.e. an piloting device) supplied with electrical energy by electrical energy supply means,
a main gas circuit comprising at least one main NO line fluidically connecting at least one NO inlet port to a main outlet orifice in order to convey an NO/N₂ mixture from said at least one NO inlet port to said main outlet orifice, said main NO line comprising NO/N₂ flowrate control means (i.e. a device) controlled by the piloting means,
a backup circuit comprising a backup NO circuit and a backup O₂ circuit, in which:
   a) the backup NO circuit comprises a backup NO line in fluidic communication with the main NO line, the backup NO line comprising a pneumatic valve making it possible to control the circulation of the NO/N₂ mixture in the backup NO line and a second solenoid valve normally in an open position, said second solenoid valve being controlled by the piloting means, and
   b) the backup O₂ circuit comprises a main O₂ line in fluidic communication with an O₂ inlet port, said main O₂ line comprising:
      a first control valve controlled by an actuation means (i.e. a device) that can be actuated by a user in order to control the circulation of the flow of oxygen in said main O₂ line, said actuation means being further configured to supply an activation signal to the piloting means in response to an actuation of said actuation means by the user,
      a second, pneumatic control valve arranged downstream of the first control valve,
   and in which the piloting means comprise at least one microprocessor and are configured, in response to the receipt of the activation signal delivered after actuation of the actuation means by the user (for example in the event of a minor fault that does not generate a total shut-down of the supply of electrical energy and/or a shut-down of the functioning of the piloting means):
      to command the second solenoid valve of the backup NO circuit to interrupt all circulation of NO/N₂ mixture through said second solenoid valve,
      to control the NO/N2 flowrate control means (i.e. a device) in such a way as to regulate the flowrate of the NO/N₂ mixture in the main gas circuit, and
      to command a first solenoid valve, arranged on the main NO line downstream of the NO/N₂ flowrate control means and on the backup NO line of the backup NO circuit downstream of the pneumatic valve and the second solenoid valve, in such a way as to direct the flow of NO/N₂ mixture coming from the NO/N₂ flowrate control means, in a downstream portion of the backup NO line connecting to the main O₂ line in order to form a common backup line in fluidic communication with a backup outlet, i.e. a secondary outlet.

According to the embodiment in question, the NO supply device or apparatus according to the invention can comprise one or more of the following features:
the piloting means (i.e. an piloting device) are configured to receive, when they are supplied with electrical energy (i.e. electric current), the activation signal delivered after actuation of the actuation means by the user.
it comprises oxygen flowrate measurement means (i.e. an oxygen flowrate measurement device) arranged downstream of the first control valve and configured to supply at least one oxygen flowrate measurement to the piloting means.
it comprises O₂ flowrate regulating means arranged downstream of the oxygen flowrate measurement means.
the main gas circuit comprises a main NO line connected fluidically to two NO inlet ports that are arranged in parallel.
the backup NO line of the backup NO circuit and the main O₂ line of the backup O₂ circuit are connected fluidically to each other at a junction site situated downstream of the O₂ flowrate regulating means of the main O₂ line and downstream of the second solenoid valve of the backup NO line.
the O₂ flowrate control means comprise a flowrate sensor or a differential pressure sensor.
the main O₂ line is configured to cooperate pneumatically with the pneumatic valve by way of a conduit section fluidically connecting the main O₂ line, downstream of the second control valve, to said pneumatic valve.
the first control valve is of the all-or-nothing type.
the first control valve is configured to control the circulation of the oxygen flow in the main O₂ line.
the first control valve is controlled by an actuation means that can be actuated by the user, for example a member of the medical personnel, when they wish to start or stop the backup circuit.
the actuation means is a rotary selector that can be actuated by the user.
it comprises NO content regulating means (i.e. a device) configured to allow a user to choose a desired NO content and supply said desired NO content to the piloting means.
it further comprises an information display screen, preferably a touch-controlled digital screen.
the NO content regulating means comprise at least one touch-activated selection key displayed on the display screen, i.e. one or more touch-activated selection keys, and configured to allow a user to set or select a desired NO content. In other words, the desired NO concentration is thus selected or set by the user’s finger pressing on the touch-activated selection key(s) displayed on the display screen.
the piloting means are configured to govern the one or more displays on the display screen.
the piloting means comprise at least one (micro)processor, for example a microcontroller.
the piloting means comprise at least one electronic board comprising said at least one microprocessor.
the piloting means comprise at least one (micro)processor implementing at least one algorithm, for example for data processing, calculation or similar.
the piloting means are configured to calculate the flowrate of NO/N₂ mixture to be supplied on the basis of the desired NO content, the oxygen flowrate measured by the oxygen flowrate measurement means and an NO content in the NO/N₂ mixture conveyed via the main NO line.
the piloting means are further configured, in response to the receipt of the activation signal, to act on the second solenoid valve in order to interrupt all circulation of NO/N₂ mixture in the backup NO line.
it comprises a first solenoid valve, controlled by the piloting means and arranged on the main NO line downstream of the NO/N₂ flowrate control means.
the first solenoid valve is arranged on, i.e. cooperates with, the backup NO line of the backup NO circuit downstream of the pneumatic valve.
the main NO line is connected fluidically to the backup NO line, downstream of the NO/N₂ flowrate control means, by way of the first solenoid valve, which is a multi-way solenoid valve, preferably an at least 3-way solenoid valve.
the first multi-way solenoid valve comprises at least one inlet path connected fluidically to the main NO line downstream of the flowrate control means, and a first outlet path connected to the main NO line, in particular to the downstream portion of the main NO line comprising the main outlet orifice, and a second outlet path connected to the backup NO line upstream of the common line comprising the secondary orifice.
the first multi-way solenoid valve cooperates with the backup NO line via a connection conduit connecting the backup NO line to one of the paths of the first solenoid valve.
the main NO line comprises at least one upstream conduit portion, preferably two upstream conduit portions arranged in parallel.
the backup NO circuit is supplied with NO/N₂ via said at least one upstream conduit portion of the main NO line, preferably the two upstream conduit portions arranged in parallel.
the backup NO circuit is connected fluidically to said at least one upstream conduit portion, preferably to the two upstream conduit portions arranged in parallel, via at least one NO inlet channel section, preferably two NO inlet channel sections arranged in parallel and connecting fluidically to the two upstream conduit portions.
the backup NO circuit connects fluidically to the main NO line, upstream of the NO/N₂ flowrate control means, in particular to said upstream conduit portions of the main NO line, so as to be supplied with NO/N₂.
the first solenoid valve is configured to direct the flow of NO/N₂ mixture:
   either in a downstream portion of the main NO line comprising the main outlet orifice,
   or in a downstream portion of the backup NO line connecting to the main O₂ line.
the first solenoid valve is a 3-way solenoid valve.
the first solenoid valve is arranged on the downstream conduit portion of the main NO line, between the NO/N₂ flowrate control means and the main outlet orifice.
the first solenoid valve is controlled by the piloting means.
the first solenoid valve is commanded by the piloting means to authorize or stop all passage of NO/N₂ mixture coming from the NO/N₂ flowrate control means, in particular to the main outlet orifice or to the junction site of the main O₂ line and the backup NO line.
the first solenoid valve comprises 3 paths and is connected to the downstream conduit portion of the main NO line and to the backup NO line of the backup NO circuit, via said 3 paths.
the junction site is situated downstream of the first solenoid valve.
the backup NO line comprises a device, with calibrated orifice, arranged downstream of the pneumatic valve.
the backup NO line of the backup NO circuit is connected fluidically to the main NO line downstream of the flowrate control means.
the backup NO line of the backup NO circuit is connected fluidically to the main NO line via the first solenoid valve, in particular a 3-way solenoid valve.
a first pressure regulator is arranged on the backup NO line upstream of the pneumatic valve.
a second pressure regulator is arranged on the main O₂ line between the second, pneumatic control valve and the oxygen flowrate measurement means.
an NO flowrate indicator device is arranged on the backup NO line upstream of the junction site of the main O₂ line and the backup NO line.
an O₂ flowrate indicator device is arranged on the main O₂ line, downstream of the O₂ flowrate regulating device or means.
the O₂ flowrate indicator device comprises a ball rotameter.
the O₂ flowrate regulator means comprise a rotary disc with calibrated orifices or the like.
the O₂ flowrate regulator means are configured to adjust the flowrate of the O₂ flow between 5 and 20 I/min.
it comprises a flowrate selection means copiloting with the O₂ flowrate regulating means in order to select a desired O₂ flowrate.
the flowrate selection means can be actuated by the user.
the flowrate selection means comprise a rotary knob or similar.
according to another embodiment, the flowrate selection means comprises a key or similar displayed on a display screen for performing selection of the O₂ flowrate.
it comprises data storage means, for example a computer memory or similar.
it comprises a rigid housing, in which all or some of the elements of the NO supply device are arranged.
it comprises a backup outlet, i.e. a backup outlet port or orifice, intended to be connected fluidically to a manual insufflation bag, preferably via a connecting conduit, such as a flexible hose.
the backup outlet is carried by a connecting connector, an outlet connector or similar.
the piloting means are configured to calculate a flowrate of NO/N₂ mixture to be supplied, corresponding to the desired NO content, and to operate the NO/N₂ flowrate control means in order to supply said calculated flowrate of NO/N₂ mixture, in particular in response to the receipt of the activation signal resulting from an actuation of the actuation means by the user..
at least one touch-activated selection key is configured to allow the user to set or select a desired NO content of between 1 and 80 ppmv.
the NO content in the NO/N₂ mixture conveyed through the main NO line is between 100 and 1000 ppmv.
the main O₂ line is configured to cooperate pneumatically with the pneumatic valve in order to authorize the passage of the NO/N₂ mixture into the backup NO line, after actuation of the actuation means by the user, that is to say in response to an actuation of the actuation means by the user.
the piloting means are further configured, in the absence of actuation of the actuation means by the user, to ensure a normal functioning of the device by piloting the NO/N₂ flowrate control means in such a way as to convey the NO/N₂ mixture through the main gas circuit between said at least one NO inlet port and the main outlet orifice.
the touch-activated selection keys allow the user to make a choice from a number of proposed NO contents, i.e. to set or select a desired NO content, for example from a number of predefined NO contents.
alternatively, the touch-activated selection keys comprise “+” and “-” keys making it possible to increase or decrease a give NO value in stages, for example 1 ppmv by 1 ppmv, or by another increment (e.g. 2 by 2, 3 by 3, 4 by 4 or 5 by 5 ppmv).
said desired NO content, which is chosen or selected by the user by acting on said at least one touch-activated selection key, is supplied to the piloting means, preferably by digital action.
the display screen provides a display in colour or in black and white.
the electrical energy supply means, i.e. means for supplying electric current, comprise means for electrical connection to the mains (110/220 V), for example an electrical cable equipped with an electric socket or similar, and/or an electric battery, preferably a rechargeable one.
the display screen is carried by the housing.
the backup outlet, i.e. a secondary outlet, such as an outlet port or orifice, is configured to be connected fluidically to a manual insufflator, such as a manual ventilation bag or bag valve mask (BVM), in particular via a flexible conduit for fluidic connection, i.e. a flexible hose or similar.
a main NO line comprises at least one upstream conduit portion, preferably two upstream conduit portions arranged in parallel.
the backup NO circuit is supplied with NO/N₂ via said at least one upstream conduit portion of the main NO line, preferably the two upstream conduit portions arranged in parallel.
the backup NO circuit is connected fluidically to said at least one upstream conduit portion, preferably to the two upstream conduit portions arranged in parallel, via at least one NO inlet channel section, preferably two NO inlet channel sections arranged in parallel and connecting fluidically to the two upstream conduit portions.
the backup NO circuit connects fluidically to the main NO line, upstream of the NO/N₂ flowrate control means, in particular to said upstream conduit portions of the main NO line, so as to be supplied with NO/N₂.
the one or the two NO inlet channel section(s) is or are supplied with NO/N₂ via the one or the two NO inlet port(s).
the NO/N₂ flowrate control means comprise first control valves, second control valves, and gas-conveying sections or sub-sections.
the piloting means are configured to operate the first control valves and the second control valves in order to direct the gas flow or gas flows in one or more sections, making it possible to obtain the desired NO dosing.
the NO/N₂ flowrate control means further comprise one or more pressure regulator devices.
the NO/N₂ flowrate control means further comprise one or more pressure sensors.

According to another aspect, the invention also relates to an NO supply device or apparatus comprising:
piloting means comprising at least one microprocessor,
a main gas circuit comprising at least one main NO line fluidically connecting at least one NO inlet port to a main outlet orifice in order to convey an NO/N₂ mixture from said at least one NO inlet port to said main outlet orifice, said main NO line comprising NO/N₂ flowrate control means controlled by the piloting means, and a first solenoid valve controlled by the piloting means and arranged on the main NO line downstream of the NO/N₂ flowrate control means,
a backup circuit comprising a backup NO circuit and a backup O₂ circuit, in which:
   a) the backup NO circuit comprises a backup NO line in fluidic communication with the main NO line, the backup NO line comprising a pneumatic valve making it possible to control the circulation of the NO/N₂ mixture in the backup NO line and a second solenoid valve controlled by the piloting means, the first solenoid valve being further arranged on the backup NO line downstream of the pneumatic valve,
   b) the backup O₂ circuit comprises a main O₂ line in fluidic communication with an O₂ inlet port, said main O₂ line comprising:
      a first control valve controlled by an actuation means that can be actuated by a user in order to control the circulation of the flow of oxygen in said main O₂ line, said actuation means being further configured to supply an activation signal to the piloting means in response to an actuation of said actuation means by the user,
      a second pneumatic control valve arranged downstream of the first control valve,
      oxygen flowrate measurement means arranged downstream of the second pneumatic control valve and configured to supply at least one oxygen flowrate measurement to the piloting means, and
      O₂ flowrate regulating means arranged downstream of the oxygen flowrate measurement means,
and NO content regulating means which are configured to allow a user to choose a desired NO content and to supply said desired NO content to the piloting means,
and in which the piloting means are configured, in response to the receipt of the activation signal resulting from an actuation of the actuation means by the user:
   to calculate a flowrate of NO/N₂ mixture to be supplied corresponding to the desired NO content chosen by the user,
   to operate the NO/N₂ flowrate control means in order to supply the calculated flowrate of NO/N₂ mixture, and
   to act on the first solenoid valve in order to allowallow the passage of the NO/N₂ flow from the downstream conduit portion of the main gas circuit, situated downstream of the NO/N₂ flowrate control means, to a downstream part of the backup NO line situated downstream of the first solenoid valve and connecting fluidically to the main O₂ line in order to form a common backup line in fluidic communication with a backup outlet.

According to another aspect, the invention also relates to an NO supply device or apparatus comprising:
piloting means, in particular with microprocessor(s),
a main gas circuit comprising at least one main NO line fluidically connecting at least one NO inlet port to a main outlet orifice in order to convey an NO/N₂ mixture from said at least one NO inlet port to said main outlet orifice, said main NO line comprising NO/N2 flowrate control means controlled by the piloting means,
a backup circuit comprising a backup NO circuit and a backup O₂ circuit, in which:
   A) the backup NO circuit comprises a backup NO line in fluidic communication with the main NO line, the backup NO line comprising a pneumatic valve making it possible to control the circulation of the NO/N₂ mixture in the backup NO line and a second solenoid valve controlled by the piloting means, and
   B) the backup O₂ circuit comprises a main O₂ line in fluidic communication with an O₂ inlet port, said main O₂ line comprising:
      a first control valve controlled by an actuation means that can be actuated by a user in order to control the circulation of the flow of oxygen in said main O₂ line, said actuation means being further configured to supply an activation signal to the piloting means in response to an actuation of said actuation means by the user,
      a second, pneumatic control valve arranged downstream of the first control valve,
      oxygen flowrate measurement means arranged downstream of the second pneumatic control valve and configured to supply at least one oxygen flowrate measurement to the piloting means, and
      O₂ flowrate regulating means arranged downstream of the oxygen flowrate measurement means,
      and in which:
      the main O₂ line is configured to cooperate pneumatically with the pneumatic valve by way of a conduit section fluidically connecting the main O₂ line, downstream of the second control valve, to said pneumatic valve, in order to allow the passage of the NO/N₂ mixture into the backup NO line in response to an actuation of the actuation means by the user,
      the backup NO line of the backup NO circuit is connected fluidically to the main NO line downstream of the NO/N₂ flowrate control means, by way of a first solenoid valve, controlled by the piloting means, arranged on the main NO line downstream of the NO/N₂ flowrate control means and on the backup NO line of the backup NO circuit downstream of the pneumatic valve, said first solenoid valve being configured to direct the flow of NO/N₂ mixture:
         either in a downstream portion of the main NO line comprising the main outlet orifice,
         or in a downstream portion of the backup NO line connecting to the main O₂ line, and
      the backup NO line of the backup NO circuit and the main O₂ line of the backup O₂ circuit are connected fluidically to each other in order to form a common backup line in fluidic communication with an backup outlet.

According to another aspect, the invention also relates to an Installation for administering NO-containing therapeutic gas to a patient (P), comprising an NO supply device or apparatus according to the invention, in particular as described above, which is fed with NO/N₂ mixture by at least one pressurized gas container and with oxygen by a pressurized oxygen container, said NO supply device or apparatus supplying:
either an NO/N₂ mixture via the main outlet orifice, to a respiratory gas source connected to a medical ventilator,
or an NO/N₂/O₂ mixture via the secondary orifice, i.e. safety orifice, to a manual insufflator or bag valve mask (BVM).

According to the embodiment in question, the installation of the invention for administering therapeutic gas can comprise one or more of the following features:
said at least one pressurized gas container contains the NO/N₂ mixture.
said at least one pressurized gas container contains an NO/N₂ mixture containing from 100 to 1000 ppmv of NO, the remainder being nitrogen.
the pressurized oxygen container contains medical oxygen.
the one or more containers are gas cylinders.
the one or more gas containers contain an NO/N₂ mixture or medical oxygen at a pressure of at least 150 bar, or at least 180 bar.
the patient circuit comprises an inhalation branch and an exhalation branch.
the inhalation branch and the exhalation branch are connected fluidically to each other via a joining piece, such as a Y-piece.
the joining piece is connected fluidically to a respiratory interface such as a tracheal probe or a breathing mask.
the inhalation branch and/or the exhalation branch comprise flexible hoses.
a gas humidifier is arranged on the respiratory gas circuit, in particular on the inhalation branch.
the inhalation branch comprises a flowrate sensor connected electrically to the NO supply device, in particular to the piloting means.
the flowrate sensor is arranged on the inhalation branch upstream of the NO injection site.
the flowrate sensor is a mass-flow sensor or a differential pressure sensor.
a gas sampling line fluidically connects the NO supply device to the respiratory gas circuit, preferably in proximity to the joining piece, i.e. the Y-piece.
the medical ventilator and the NO supply device are fed electrically by electrical energy supply means, that is to say at least one source of electric current, in particular means for electrical connection to the mains (110/220 V), for example an electrical cable equipped with an electric socket or similar, and/or an electric battery, preferably a rechargeable one.
the NO supply device comprises a backup outlet, i.e. a backup outlet port or orifice, connected fluidically to a manual insufflation bag, preferably via a connecting conduit, such as a flexible hose.

According to yet another aspect, the invention also relates to a method for treating a person, called a patient, suffering from a disease or a medical condition that causes acute pulmonary hypertension, in particular pulmonary vasoconstriction, in adults, adolescents or children, including neonates and babies, for example for treating persistent pulmonary hypertension of the newborn (PPHN) in a neonate or baby, infant or similar, or pulmonary hypertension in a person undergoing heart surgery, in which there is administered by inhalation, to said patient requiring it, a gaseous mixture containing oxygen (preferably >21% by volume), nitrogen and NO (preferably <100 ppmv), said gaseous mixture being supplied by an installation according to the invention which administers therapeutic gas and comprises an NO supply device or apparatus according to the invention, in particular as described above and/or below, which NO supply device is fed with an NO/N₂ mixture by at least one pressurized gas container, preferably several pressurized gas containers such as NO/N₂ cylinders, and with oxygen by at least one pressurized oxygen container, such as an O₂ cylinder, and which NO supply device can supply: either an NO/N₂ mixture, via the main outlet orifice, to the respiratory gas circuit connected to a medical ventilator of the installation according to the invention for administration of therapeutic gas, or an NO/N₂/O₂ mixture, via the secondary orifice, to a manual insufflator, in particular a manual insufflation bag or bag valve mask (BVM).

### BRIEF DESCRIPTION OF THE DRAWINGS

The invention will now be better understood from the following detailed description given as a non-limiting example and with reference to the appended figures, in which:

FIG. 1 shows an embodiment of an installation for administering therapeutic gas to a patient P, incorporating an NO supply device,

FIG. 2 shows schematically an embodiment of the backup circuit of an NO supply device according to the prior art.

FIG. 3 shows schematically an embodiment of the internal architecture of an NO supply device according to the invention.

FIG. 4 shows a detailed diagram of the NO/N₂ flowrate control means of the device of FIG. 3.

### DESCRIPTION OF THE PREFERRED EMBODIMENTS

FIG. 1 shows schematically an embodiment of an installation 100 for administering therapeutic gas, i.e. a gaseous mixture based on NO, to a patient P, incorporating an NO supply device or apparatus 1, such the one according to the present invention.

More precisely, it here comprises two pressurized gas containers 5 which are arranged in parallel, each containing a gaseous mixture of NO and nitrogen (N₂), i.e. an NO/N₂ mixture, typically containing from 250 to 1000 ppmv of NO and nitrogen (N₂), at a pressure of 180 bar or more, for example an NO/N₂ mixture containing 450 ppmv or 800 ppmv of NO. Such gas containers 5 are commonly called NO cylinders 5.

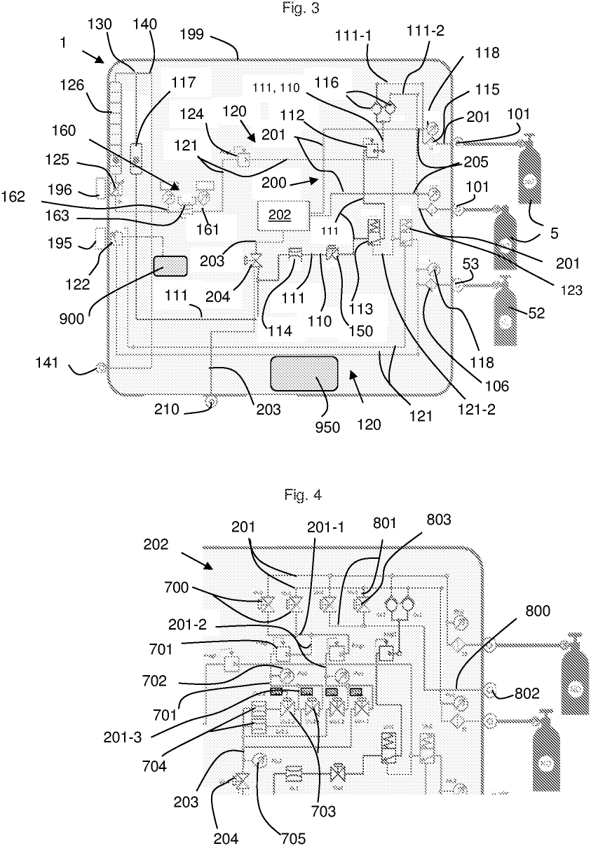

The NO cylinders 5 supply the NO/N₂ mixture to an NO supply device 1, such as the one according to the invention, of which the internal architecture is illustrated in FIG. 3 and FIG. 4. They are fluidically connected to the gas supply device 1 by NO supply lines 50, i.e. gas channels, such as flexible hoses or similar. Each NO supply line 50 is connected to an NO inlet port 101 of the NO supply device 1 in order to feed a main gas circuit 200 inside the housing 199 of the NO supply device 1 (cf. FIG. 3).

The NO supply device 1 also comprises an oxygen inlet port 53 connected fluidically, via an oxygen admission line 51, such as a flexible hose or similar, to an oxygen source, for example a pressurized oxygen container 52, typically an O₂cylinder, or, alternatively, to the hospital network, i.e. an oxygen admission duct arranged in the hospital building where the patient P is being treated.

The NO cylinders 5 and the O₂ cylinder 52 are equipped with a gas distribution valve 55, preferably incorporating means (i.e. a device) for pressure release, that is to say an integrated pressure regulator (IPR), in such a way as to be able to control the flowrate and/or the pressure of the gas that they deliver. The gas distribution valve 55 is preferably protected against impacts by a protective cap.

Furthermore, the installation 100 also comprises a medical ventilator 2, that is to say a respiratory assistance apparatus, supplying a flow of respiratory gas containing at least 21% by volume of oxygen, such as air or an oxygen/nitrogen (N₂/O₂) mixture, to the patient P.

The medical ventilator 2 is fluidically connected to the patient P via a respiratory gas circuit 3, which is here of the type with two respiratory branches 30, 31 given that it comprises an inhalation branch 30, that is to say a gas supply line, serving to feed the respiratory gas to the patient P, and an exhalation branch 31 serving to recover the CO₂-enriched gas exhaled by the patient P.

The two respiratory branches 30, 31 are typically flexible hoses made of polymer or similar. The two respiratory branches 30, 31 are, on the one hand, connected to the medical ventilator 2 and, on the other hand, connected to each other at a junction piece 32, typically a Y-piece, which is in fluidic communication with a respiratory interface 4 supplying the gas to the patient P, for example a tracheal probe or the like.

Of course, the medical ventilator 2 and the NO supply device 1 are normally powered electrically by one or more sources of electric current, particularly their components requiring electrical energy to function, in particular the piloting means 900 (i.e. a device) of the NO supply device 1 and the system for controlling the medical ventilator 2, i.e. an electronic board with one or more microprocessors, or any other component, in particular the internal motorized turbine which supplies the flow of air or similar, i.e. the respiratory gas. The source of electric current can be the mains (110/220 V) and/or an electric battery, preferably a rechargeable one.

As will be seen, the NO supply device 1 makes it possible to inject the NO/N₂ mixture into the inhalation branch 30, via an NO injection conduit 11 opening into the inhalation branch 30 at an injection site 8, so as to there cause mixing of the flow of NO/N₂ and of the flow of respiratory gas containing at least 21% of O₂, i.e. air or an oxygen/nitrogen mixture, delivered by the medical ventilator 2.

The NO supply device 1 comprises a main outlet orifice 210 situated at the mouth of its main gas circuit 200 through which the flow of NO/N₂ leaves the housing 199 of the NO supply device 1 and enters the NO injection conduit 11. The NO injection conduit 11 is connected fluidically to the main outlet orifice 210, for example via a connector or similar.

The therapeutic gaseous mixture obtained thus contains oxygen (>21% by volume), nitrogen and a variable and adjustable concentration of NO, typically of between 1 and 80 ppmv, on account of the dilution that takes place during the mixing of the flows of gas. Of course, inevitable impurities may be present in the gas, but they are undesirable, particularly when the flow of gas coming from the ventilator 2 is atmospheric air rather than an O₂/N₂ mixture.

Advantageously, a gas humidifier 6 is also provided, which is arranged here on the inhalation branch 30 downstream of the injection site 8 and serves to humidify the flow of therapeutic gas, e.g. an NO/N₂/O₂ mixture, by addition of water vapour, before the mixture is inhaled by the patient P, by which means it is possible to avoid or limit drying out of the airways of the patient P during his or her treatment by inhalation of gas. According to another embodiment, the gas humidifier 6 could also be arranged upstream of the injection site 8. Depending on the circumstances, the exhalation branch 31 serving to recover the exhaled gases rich in CO₂ can comprise one or more other optional components, for example a device for eliminating the CO₂, i.e. a CO₂ trap, such as a hot pot or the like, allowing the removal of the CO₂ present in the gases exhaled by the patient, or a filter or the like.

As can be seen in FIG. 1, there is also provided on the inhalation branch 30, upstream of the injection site 8, a flowrate sensor 7, for example a mass-flow sensor or a differential pressure sensor,connected to the NO supply device 1, in particular to the piloting means 900 of said NO supply device 1, via a flowrate measurement line 71 for respiratory gas, serving to measure the flowrate of gas, coming from the ventilator 2, within the inhalation branch 30. By determining this flowrate of the ventilator (Qv), it is possible in particular to regulate the passage of the NO through the NO supply device 1, in particular to be able to choose the flowrate of NO/N₂ mixture to be injected on the basis of the desired NO content, the composition of the NO/N₂ mixture coming from the cylinders, and the flowrate of gas (i.e. air or air/O₂) coming from the ventilator 2.

Furthermore, it is also possible to provide a gas-sampling line 33 fluidically connecting the NO supply device 1 to the respiratory gas circuit 3, preferably in proximity to the Y-piece 32, so that samples of gas can be taken and their compliance with the desired gaseous mixture that is to be administered to the patient P can be verified.

More precisely, the NO supply device 1 comprises an internal main gas circuit 200 through which the NO/N₂ mixture entering via the gas inlet port(s) 101 is conveyed as far as the NO injection site 11. This main gas circuit 200 comprises NO/N₂ flowrate control means 202 (i.e. a device) (cf. FIG. 3 and FIG. 4), for example valves, calibrated orifices, etc., which are controlled by the piloting means 900 of the NO supply device 1, typically one or more microprocessors arranged on an electronic board, the function of which is explained below. All of these components are arranged in a housing 199, that is to say a rigid external shell.

Moreover, the NO supply device 1 comprises a backup circuit 110 designed to deliver an adjustable O₂ flowrate and a fixed NO flowrate, so as to be able to ensure a supply of NO even in the event of fault or the like, as is set out in detail below.

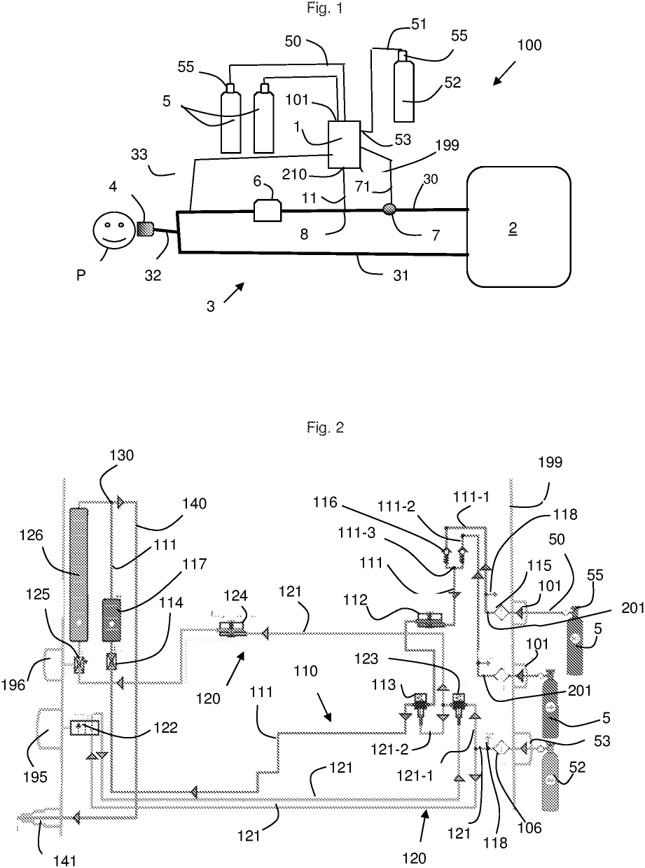

Thus, FIG. 2 shows schematically an embodiment of the backup circuit 110 or secondary circuit of the traditional NO supply device 1. It should first of all be noted that the main gas circuit 200 of the NO supply device 1, which conveys the NO/N₂ mixture during the normal operation of the device 1, that is to say in the absence of a fault or the like, is not shown in FIG. 2, so as not to needlessly complicate the diagram and make it more difficult to understand. Nonetheless, this main gas circuit 200, which is arranged in the housing 199, comprises, as is illustrated in FIG. 3 or FIG. 4, a main NO line 201, 203 comprising at least one upstream conduit portion 201, namely here two upstream conduit portions 201 arranged in parallel, since two NO cylinders 5 are connected to the NO supply device 1, as is illustrated in FIG. 2 to FIG. 4, and moreover at least one downstream portion 203.

As can be seen, the backup circuit 110, 120, the functioning of which may be entirely pneumatic, is also arranged in the housing 199 and comprises a backup NO circuit 110 and a backup O₂ circuit 120, which each comprise gas channels, passages or conduits for transporting the various gases or gaseous mixtures in the housing 199.

The backup NO circuit 110 is fed with NO/N₂ via at least one NO inlet channel section 111-1, 111-2, namely here two NO inlet channels sections 111-1, 111-2 arranged in parallel, themselves fed via the two NO inlet ports 101, to which the NO cylinders 5 are connected via the supply line 50, as has been explained above.

The one or more inlet channel sections 111-1, 111-2 are connected fluidically to the upstream conduit portions 201 (partially shown in FIG. 2) downstream of a filter 115, which is situated immediately downstream of each NO inlet port 101 in the one or more upstream conduit portions 201, as is shown in detail in FIG. 3.

A non-return valve 116 is arranged in each inlet channel section 111-1, 111-2. These two inlet channel sections 111-1, 111-2 join downstream of the non-return valves 116 in order to form a common backup NO line 111, starting from an injection site 111-3.

Each upstream conduit portion 201 further comprises pressure measurement means 118, such as a pressure sensor with strain gauge (or silicon pressure sensor) or the like, serving to verify the existence of a pressure in the upstream conduit portion 201 reflecting the presence of a connected cylinder, and thus also serving to verify the quantity of gas that the latter contains, so as to decide whether to change it (if the cylinder is almost empty) and switch to the other gas cylinder. The pressure measurement means 118 is situated downstream of the filter 115.

Of course, according to another embodiment, the device 1 can comprise just one NO inlet port 101 and therefore a single upstream conduit portion 201 and a single inlet channel section 111-1. In this case, it is possible for just one oxygen admission line 51, for example a flexible hose or similar, to be fluidically connected there, which line is fed with NO/N₂ mixture via one or more NO cylinders 5.

The backup NO line 111 makes it possible to convey the NO/N₂ mixture under pressure coming, via the inlet channel sections 111-1, 111-2, from the upstream conduit portions 201 fed from the NO cylinders 5 delivering the NO/N₂ mixture at a pressure of the order of 3 to 6 bar relative at the outlet of the integrated pressure regulator 55.

It will also be seen that the backup NO line 111 further comprises a first pressure regulator 112, downstream of the junction site 111-3, in order to reduce or control the pressure of the NO/N₂ mixture, for example in order to deliver a reduced pressure equal to about 3.2 bar relative, and a pneumatic valve 113 for controlling the circulation of the NO/N₂ mixture. The pneumatic valve 113 is situated downstream of the first pressure regulator 112, in the direction of circulation of the gas, knowing that the NO/N₂ gaseous mixture circulates in the direction from each gas inlet port 101 to the pressure regulator 112.

The opening of the pneumatic valve 113, hence the passage of the NO/N₂ flow, is controlled by the pressure of the oxygen flow conveyed through the main O₂ line 121 via the second conduit section 121-2, as explained below.

Downstream of the pneumatic valve 113, the backup NO line 111 comprises a device with a calibrated orifice 114 or similar, making it possible to regulate the flowrate of the gas, i.e. of the NO/N₂ mixture, and an NO flowrate indicator device 117 for checking that the NO/N₂ mixture is indeed equal to the set value expected, for example of the order of 230 ml/min.

In addition, the backup oxygen circuit 120 for its part comprises a main O₂ line 121 for conveying oxygen in the housing 199, which line is fed through the O₂ inlet port 53 to which the O₂ cylinder 52 is connected, via the feed line 51 which carries the gaseous oxygen at a pressure of typically between 3 and 6 bar.

The main O₂ line 121 connects, at a junction site 130, to the backup NO line 111 downstream of the NO flowrate indicator device 117 so as to cause mixing of the flow of oxygen at an adjustable flowrate, as is explained below, and the flow of NO/N₂ which has a fixed flowrate of the order of 230 ml/min, for example.

As can be seen in FIG. 2, the main O₂ line 121 also comprises a filter 106, which is arranged immediately downstream of the O₂ inlet port 53, and also a first control valve 122 and a second, pneumatic control valve 123. It also comprises a pressure measurement means 118, such as a pressure sensor with a strain gauge, as explained above.

The first control valve 122, which is typically of the all-or-nothing type, makes it possible to control the circulation of the flow of oxygen in the main O₂ line 121. It is controlled by an actuation means 195, such as a rotary selector switch, a pushbutton, a selection key or the like, which can be actuated by the user, such as a member of the medical personnel, when he or she wishes to start or stop the backup circuit 110, 120.

A first conduit section 121-1 connects fluidically to the main O₂ line 121, between the O₂ inlet port 53, in particular downstream of the filter 106, and the first control valve 122. The first conduit section 121-1 makes it possible to pneumatically operate the second, pneumatic control valve 123.

For as long as the first control valve 122 is closed, the gaseous pressure which is exerted in the portion of the main O₂ line 121 situated upstream of the first control valve 122, and thus also in the first conduit section 121-1, will act on the second, pneumatic control valve 123, keeping it closed, which will prevent any passage of oxygen through this second control valve 123.

When the user actuates the actuation means 195, the first control valve 122 opens and thus allows the passage of the gas in the direction of the second control valve 123. On account of the pressure of the oxygen reaching the second control valve 123, a sufficient force will be applied to make it possible to open the control valve 123, as a result of the pressure provided by the first conduit section 121-1, which will then open the second control valve 123, and thus allow the oxygen to pass into the downstream part of the oxygen circuit 120 which is situated downstream of the second control valve 123.

The oxygen then continues its route in the main O₂ line 121, passing through a second pressure regulator 124 in order to reduce or control the pressure of the flow of oxygen, for example in order to obtain a pressure of 1.6 bar absolute relative. The flowrate of the flow of O₂ can then be adjusted, for example between 5 and 20 I/min, by means (i.e. a device) 125 for regulation of the O₂ flowrate, such as a rotary disc with calibrated orifices or the like, arranged on the main O₂ line 121 downstream of the second pressure regulator 124.

The desired flowrate of O₂ can be selected by the user via a flowrate selection means 196, such as a rotary knob or the like, supported by the housing 199 of the device (or, according to another embodiment, selection of a flowrate of O₂ via a key or the like displayed on a display screen 950), which flowrate selection means 196 cooperates with the means 125 for regulation of the O₂ flowrate.

The flowrate of oxygen can then be checked via an O₂ flowrate indicator device 126, such as a ball rotameter or the like, arranged downstream of the device or means 125 for regulation of the O₂ flowrate. The flow of oxygen obtained can then mix with the flow of NO/N₂ starting from the junction site 130, where the main O₂ line 121 connects to the backup NO line 111, as has already been mentioned.

Since this backup circuit 110, 120 is entirely pneumatic, in order to control the circulation of NO/N₂ within the backup NO line 111, a second conduit section 121-2 is provided, which connects fluidically to the main O₂ line 121 between the second control valve 123 and the second pressure regulator 124. This second conduit section 121-2 controls the pneumatic valve 113 of the backup NO circuit 110, by virtue of the oxygen pressure that it contains.

More specifically, for as long as the second control valve 123 is closed, the pressure of the flow of oxygen is not exerted in the second conduit section 121-2, and therefore the pneumatic valve 113 of the NO circuit 110 also remains closed.

On the contrary, when the second control valve 123 opens after actuation of the actuation means 195 by the user and after opening of the first control valve 122, as has been explained above, the flow of pressurized oxygen will be able to pass through the second control valve 123, then spread and advance downstream thereof, in particular into the second conduit section 121-2, in order then to act pneumatically on the pneumatic valve 113 of the NO circuit 110 and open it, thus opening up the passage for the NO/N₂ mixture and its circulation in the downstream part of the backup NO line 111 situated downstream of the pneumatic valve 113.

The first control valve 122, the second control valve 123 and the pneumatic valve 113 are, for example, controlled pneumatic valves of the shutter and spring type.

In other words, the activation of the actuation means 195 by the user leads to quasi-synchronized and/or quasi-simultaneous release of the flows of NO/N₂ and O₂ mixture within the backup NO circuit 110 and the backup oxygen circuit 120, thus resulting in a mixing thereof starting from, and downstream of, the junction site 130, that is to say in the common backup line 140 which opens at a backup outlet 141, i.e. a secondary outlet, which for example is supported by a connection connector or the like, to which can be connected a manual gas insufflator, that is to say a manual ventilation bag or similar.

It will be understood that the operation of this backup or secondary gas circuit 110, 120 is entirely pneumatic, since it uses only gas conduits and pneumatic valves in order to control the flows of gas.

However, as has already been explained, an entirely pneumatic circuit of this type is not ideal, since the NO concentration of the gaseous mixture obtained (i.e. NO/N₂ + O₂) starting from the junction site 130, and thus in the common backup line 140 situated downstream, varies according to the flowrate of oxygen regulated by the user, since the flowrate of NO is fixed, whereas that of oxygen is adjustable.

Thus, for a NO/N₂ gaseous mixture with 800 ppmv of NO (remainder nitrogen), the NO concentration varies between 8 and 32 ppmv for a flowrate of oxygen of between 5 and 20 I/min, whereas, for a NO/N₂ mixture with 450 ppmv of NO, the NO concentration varies between 4.5 and 18 ppmv for the same flowrate of oxygen.

However, having an NO concentration which varies according to the chosen flowrate of O₂ is not acceptable, since it is necessary to be able to provide the patient with a given dose, that is to say a fixed quantity of NO corresponding to a concentration which is efficient for treating the patient’s pathology, in particular within the context of ventilation of the patient by a manual ventilation bag in place of normal ventilation by the medical ventilator 2.

It is therefore necessary to be able to make the NO concentration of the obtained gaseous NO/N₂/O₂ mixture independent of the flowrate of oxygen, when the backup mode is activated by the user and ventilation of the patient by means of a manual ventilation bag has to be put into effect.

For this purpose, there is provided the improved NO supply device 1 as illustrated in FIG. 3 and FIG. 4, which show schematically an embodiment of the internal architecture of an NO supply device 1 according to the present invention.

Firstly, the NO supply device 1 of FIG. 3 comprises the entirely pneumatic elements of the backup gas circuit 110, 120 of FIG. 2, which bear the same references and are therefore not explained again hereinbelow.

Next, as can be seen in FIG. 3, the NO supply device 1 of the invention also comprises the internal main gas circuit 200 which, during normal operation of the device 1, is used to supply the NO/N₂ mixture via the NO injection conduit 11 to the inhalation branch 30 of the ventilation circuit 3, which is connected to and supplied by the medical ventilator 2 with respiratory gas, i.e. air or an N₂/O₂ mixture, as has been explained above in connection with FIG. 1.

The main gas circuit 200 comprises at least one upstream conduit portion 201 i.e. in this case two upstream conduit portions 201 to which the inlet channel sections 111-1, 111-2 connect fluidically, at a connection site 205 situated between the filter 115 and the non-return valve 116 of each inlet channel section 111-1, 111-2, as shown in FIG. 3. Of course, if only one inlet channel section 111-1 is provided, according to the embodiment considered, as explained above, then only one upstream conduit portion 201 is necessary.

The one or more upstream conduit portions 201 make it possible to convey the NO/N₂ mixture, obtained from the inlet channel sections 111-1, 111-2, as far as means (i.e. a device) 202 for NO/N₂ flowrate control, which make it possible to control the flowrate of the flow of NO/N₂ mixture that then has to be supplied to the injection conduit 11. An embodiment of the NO/N₂ flowrate control means 202 is shown in detail in FIG. 4.

After its passage into the NO/N₂ flowrate control means 202, the gaseous flow is conveyed through a downstream conduit portion 203 of the main NO line 201, 203, which brings it and supplies it to the injection conduit 11, via the main outlet orifice 210 which is at the outlet end of the downstream conduit portion 203 of the main gas circuit 200.

A first solenoid valve 204 of the 3-way type is arranged on the downstream conduit portion 203 of the main NO line 201, 203, between the NO/N₂ flowrate control means 202 and the main outlet orifice 210, that is to say it is connected by 2 of these 3 ways to the downstream conduit portion 203, so as to control the circulation of the gas in the downstream conduit portion 203. It is also connected, via its third way, to the backup NO line 111, downstream of the pneumatic valve 113, which itself is situated downstream of the first pressure regulator 112, taking into consideration the direction of circulation of the gas, i.e. the NO/N₂ mixture, in the backup NO line 111.

Furthermore, as shown in FIG. 3, the backup NO line 111 comprises a second solenoid valve 150 of the 2-way type, which is normally in the open position, that is to say it allows the gas to circulate within the backup NO line 111, and the device with a calibrated orifice 114 or the like which makes it possible to regulate the gaseous flowrate, i.e. the flowrate of the NO/N₂ mixture, which in FIG. 2 is arranged in proximity to the NO flowrate indicator device 117. The second solenoid valve 150 is thus situated between the pneumatic valve 113 and the first solenoid valve 204 with 3 ways.

The second solenoid valve 150 is normally in the open position, but it can be controlled by the piloting means 900, in particular in the case of activation of the backup mode in the event of a minor fault, as explained below, or if it is necessary to ventilate a patient by means of the manual insufflator in order to carry out alveolar recruitment manoeuvres.

According to another embodiment (not shown), the first solenoid valve 204 with 3 ways and the second solenoid valve 150 with 2 ways can be replaced by a single solenoid valve with 5 ways, which would be installed instead of, and in the place of, the first solenoid valve 204 with 3 ways (the second solenoid valve 150 would then be eliminated).

In addition, the NO supply device 1 according to the invention finally comprises means (i.e. a device) 160 for oxygen flowrate measurement, which are arranged on the main O₂ line 121 between the second pressure regulator 124 and the O₂ flowrate regulating means 125.

These oxygen flowrate measurement means 160 can comprise a flowrate sensor or a differential pressure sensor, the pressure ports 161, 162 of which are connected upstream and downstream of a flow restrictor 163, such as a Venturi system, a calibrated orifice or the like, in order to measure a pressure differential (ΔP), i.e. a pressure drop, making it possible to deduce therefrom a flowrate of oxygen (Q_{O2}).

FIG. 4 shows a detailed embodiment of the NO/N₂ flowrate control means 202 which are used to control the flowrate of NO/N₂ within the main gas circuit 200 arranged in the housing 199 of the NO supply device 1.

As can be seen, the two upstream conduit portions 201 of the main gas circuit 200 join up in order to form a common section 201-1 conveying the NO/N₂mixture. Each upstream conduit portion 201 comprises a first control valve 700, which is arranged upstream of the site of junction of the two upstream conduit portions 201 and which is used to allow or stop the circulation of the flow of NO/N₂ mixture supplied by one or the other of the upstream conduit portions 201.

The common section 201-1 then divides into two secondary sections 201-2 arranged in parallel with each other, which each comprise a pressure regulator device 701 and a pressure sensor 702.

The two secondary sections 201-2 divide in turn into a plurality of sub-sections 201-3, which are also arranged in parallel with one another, i.e. in this case two sub-sections 201-3. Each sub-section 201-3 comprises a second control valve 703. The sub-sections 201-3 also comprise additional flowrate control means (i.e. a device) 704, for example one or more calibrated orifices or the like, making it possible to adjust the flowrate to a desired flowrate value.

Of course, the NO/N₂ flowrate control means 202 can comprise more sub-sections 201-3, which in particular are each equipped with a control valve 703 and additional flowrate control means 704, typically a calibrated orifice, in order to make it possible to provide a greater variety of NO flowrates, if necessary. Preferably, the control valve(s) 703 is/are a valve/valves of the all-or-nothing type (AON). The flowrate of gas circulating therein is fixed and depends on the pressure of the pressure regulator 701, i.e. a pressure reducer, and on the cross section of the calibrated orifice.

In fact, the piloting means 900 of the NO supply device 1, such as an electronic board bearing one or more microprocessors which implement one or more algorithms, govern the first control valves 700 and the second control valves 703, in order to direct the gases into the appropriate sections, making it possible to obtain the desired dose of NO.

The desired NO content is chosen by the user via means (i.e. a device) (not shown) for selection of the NO content, which means are arranged on the device, for example one or more keys, cursors, selection knobs, in particular a rotary knob or the like, which make it possible to select or set a desired NO content.

Advantageously, the NO supply device 1 according to the invention is equipped with an information display screen 950, typically a digital screen, preferably a touchscreen, in colour or black and white, which is configured to display the value of the desired NO content and/or other information, such as the NO content in the NO cylinders 5, or also the flowrate values of NO or O₂, and for example also the concentration of NO₂, the flowrate delivered by the ventilator, etc.

According to one embodiment, the information display screen 950 is a digital screen with touch control, and the means for selection of the NO content are keys with selection by touch actuation, which are displayed on the digital display touchscreen 950. The desired NO concentration is thus selected or set by the user pressing his or her finger on the keys for selection by touch actuation displayed on the display screen 950.

Once the NO content has been selected, the piloting means 900 of the NO supply device 1 calculate the flowrate of NO/N₂ mixture to be supplied, in particular depending on other parameters, such as the NO content of the NO/N₂ mixture obtained from the cylinders 5, and also the flowrate of respiratory gas (typically air or air/O₂) supplied by the ventilator 2, and then determine which control valves 700, 703 must be opened or closed in order to direct the gases into the sections suitable for obtaining the dose of NO that has been set.

The sub-sections 201-3 then join together, upstream of the first solenoid valve 204 with 3 ways, in a single line which forms the downstream conduit portion 203 of the main gas circuit 200. This can comprise a pressure sensor 705 or the like, upstream of the first solenoid valve 204, in order optionally to be able to correct the flowrate, which depends on the variation in pressure (ΔP) of the gas when it passes through the additional flowrate control means 704, typically one or more calibrated orifices.

In addition, it will also be seen that the NO supply device 1 according to the invention also comprises a purging line 800, which communicates with the exterior via a purging orifice 802 arranged in the casing 199. The purging line 800 branches into two purging sections 801 which are connected to the two upstream conduit portions 201, in order to be able to carry out gaseous purging of these two upstream conduit portions 201. Each purging section 801 comprises a purging valve 803 which is controlled by the piloting means 900, so as to be able to eliminate all the types of NO₂ which can form in the residual gas present in the channels by means of oxidation of the NO molecules by oxygen, when the device 1 is not piloting, i.e. when it is not being used.

In normal operation, i.e. when the user has not actuated the actuation means 195, the NO/N₂ mixture is conveyed in the main gas circuit 200 of the NO supply device 1 according to the invention, via the NO/N₂ flowrate control means 202, from one of the gas inlet ports 101 as far as the main outlet port 210 which supplies the NO/N₂ mixture to the NO injection conduit 11, so as to be able then to inject it at the desired concentration of NO into the inhalation branch 30 of the ventilation circuit 3 connected to the medical ventilator 2, as has been explained above with reference to FIG. 1 and FIG. 4.

In this case, the second solenoid valve 150 with 2 ways is in the “normally open” position, i.e. it does not interrupt the passage of gas in the backup NO line 111, and the first solenoid valve 204 with 3 ways is in the “normal operation” position which allows the passage of the gaseous flow into the downstream conduit portion 203 as far as the main outlet orifice 210.

The first solenoid valve 204 is controlled by the operating or piloting means 900 in order to allow or stop any passage of NO/N₂ mixture obtained from the means 202 for control (i.e. regulation) of the flowrate of NO.

In the event of a serious or severe fault of the NO supply device 1 according to the invention resulting in a loss of electrical energy and/or stoppage of the function of the piloting means 900 of the device 1, or of the medical ventilator 2, in particular involving software or another program or piloting algorithm implemented by one or more microprocessors of said piloting means, the second solenoid valve 150 with 2 ways remains in the “normally open” position, and the first solenoid valve 204 with 3 ways remains in the “normal operation” position, so as to allow circulation of the gaseous flow in the downstream conduit portion 203, as far as the main outlet orifice 210. After actuation of the actuation means 195 by the user, this makes possible a transition to a backup mode of an entirely pneumatic type, as has been explained with reference to with FIG. 2 This therefore results in the delivery, by the backup NO line 111, downstream from the NO flowrate indicator device 117, of a flow of NO/N₂ at a fixed flowrate of the order of 230 ml/min for example, which is mixed (at 130) with the flow of oxygen with an adjustable flow rate coming from the main O₂ line 121. The backup gaseous mixture thus obtained, formed of NO/N₂/O₂, can then be conveyed through the common line 140 as far as the backup outlet 141, i.e. secondary outlet, which then supplies this backup gaseous mixture to a manual ventilation bag, also known as a manual insufflator or or bag valve mask. Then, the ventilation of the patient is thus carried out via the manual ventilation bag, in order to ensure a supply of NO to the patient despite the serious fault or the like.

On the other hand, if the fault of the NO supply device 1 according to the invention is a minor fault that does not result in a total loss of electrical energy and/or a stoppage of the function of the piloting means 900 of the device 1, and/or if the medical staff decide to carry out ventilation of the patient via a manual ventilation bag while desiring to comply with the dose of NO, then the NO supply device 1 according to the invention, and more particularly the piloting means 900 and the display screen 950, continue to be supplied with electrical energy by the electrical energy supply means, and therefore the piloting means 900 continue to be able to operate.

In this case, the user wishing to put the device 1 into backup mode or into the mode of ventilation by bag valve mask, in order to ensure a supply of gas to the patient via a manual ventilation bag or bag valve mask, will, as previously, actuate the actuation means 195, which will then give rise to opening of the first control valve 122, thus allowting circulation of the flow of oxygen in the main O₂ line 121 as far as the second control valve 123, which will thus also open and allow the oxygen to pass into the downstream part of the oxygen circuit 120, situated downstream of the second control valve 123, as has been explained above with reference to FIG. 2.

The oxygen flowrate measurement means 160, which are arranged on the main O₂ line 121 downstream of the second pressure regulator 124, will then be able to measure the oxygen flowrate (QO₂) and transmit this measurement of flowrate of O₂ (i.e. signal or value) to the piloting means 900, in order to make it possible to calculate the flowrate of NO/N₂ to be supplied, as is explained below.

The flow of oxygen can then continue its route as far as the junction site 130, where the O₂/NO/N₂ mixture takes place, via the O₂ flowrate control means 125 and the O₂ flowrate indicator device 126, such as a ball rotameter or the like, as has already been explained.

However, in this case, the actuation means 195 is connected electrically to the piloting means 900 and is configured to supply the latter with actuation information, namely an activation signal corresponding to the position of the actuation means 195, namely in backup mode (i.e. backup activated) or in normal mode (i.e. backup not activated). This activation signal is supplied electrically to the piloting means 900 of the NO supply device 1 according to the invention, which receives and processes it in order to determine whether the actuation means 195 has been actuated by the user in order to trigger the backup mode or the mode of ventilation by bag valve mask, for the purpose of carrying out ventilation of the patient via a manual ventilation bag connected to the secondary outlet, i.e. the backup outlet 141, of the NO supply device 1 according to the invention, for example via a flexible tube.

If this is the case, the piloting means 900 of the NO supply device 1 according to the invention which continue to be supplied with electrical energy (i.e. electric current) retroact, in response to this signal, on the NO/N₂ flowrate control means 202, in order to supply the NO/N₂ mixture at the desired flowrate, and on the first solenoid valve 204 with 3 ways, in order to allow the passage of the flow of NO/N₂ from the downstream conduit portion 203 of the main gas circuit 200 to the downstream part of the backup NO line 111 which is situated downstream of the first solenoid valve 204 and conveys the flow of NO/N₂ via the NO flowrate indicator device 117, as far as the mixture site 130, where the main O₂ line 121 and the backup NO line 111 join, as has already been explained.

Thus, with the NO/N₂ mixture being produced within the NO/N₂ flowrate control means 202, it is possible to set the most suitable flowrate of NO. This flowrate of NO/N₂ mixture to be supplied by the NO/N₂ flowrate control means 202 can be calculated by the piloting means 900 of the NO supply device 1 according to the invention from the desired final content of NO, from the composition of the NO/N₂ mixture in the cylinders 5, and from the flowrate of oxygen measured by the oxygen flowrate measurement means 160, which are arranged on the main O₂ line 121 downstream of the second pressure regulator 124.

In this case, the desired final content of NO is thus chosen by the user via the NO content regulating means arranged on the device, preferably one or more digital keys actuated by the fingers and displayed on the digital display screen 950, which also continues to be supplied with electric current.

Being able to use an NO/N₂ mixture produced within the NO/N₂ flowrate control means 202, in the case of activation of the backup circuit and in the absence of a complete failure of the device 1, that is to say in the case of a minor fault, and/or if it is desired to ventilate the patient via a manual ventilation bag, has the advantage of guaranteeing greater precision of the concentration of NO of the backup mixture supplied to the patient, since said final content of NO can be regulated by the user via the NO content regulating means (i.e. choice or selection), and the piloting means 900 use this regulated NO content value, the concentration of NO in the NO/N₂ mixture supplied by the NO cylinders 5, and the value of O₂ flowrate measured, in order to determine the flow rate of NO/N₂ to be supplied, and they act accordingly on the NO/N₂ flowrate control means 202, in particular the first control valves 700 and the second control valves 703, so as to supply the appropriate flowrate of NO/N₂ by controlling the passage of the gas, in particular into the secondary sections 201-2, the pressure regulator device 701, the two secondary sections 201-2, and the sub-sections 201-3 comprising the additional flowrate control means 704, for example calibrated orifices or the like, thus making it possible to adjust the flowrate to a desired flowrate value.

In other words, by incorporating the oxygen flowrate measurement means 160 on the main O₂ line 121, downstream of the second pressure regulator 124, which measure the oxygen flowrate (QO₂) and transmit this O₂ flowrate measurement to the piloting means, the latter can calculate an NO/N₂ flowrate setpoint to be supplied by the NO/N₂ flowrate control means 202, also taking into account not only the NO setpoint which is regulated by the user, but also the concentration of the NO in the NO/N₂ mixture obtained from the NO cylinders 5.

This results in being able to obtain from the mixture site 130 a final mixture with a precise content of NO, since the flowrate of NO/N₂ mixture is no longer fixed (e.g. equal to 230 ml/min), but is adjustable according to the other parameters that have an influence.

However, in this case, it is essential to prevent the flow of oxygen, which passes through the second control valve 123 and acts on the pneumatic valve 113, from giving rise to an excessive supply of NO via the backup NO line 111, through the second solenoid valve 150 with 2 ways. For this purpose, when they receive the activation signal obtained from the actuation means 195, which corresponds to transition to backup mode, the piloting means 900 of the NO supply device 1 according to the invention are also configured to act further on the second solenoid valve 150 with 2 ways, which is normally in the open position, in order to close it and thus prevent the NO/N₂ mixture from being able to circulate within the backup NO line 111, and supply the downstream part of the backup NO line 111 which is situated downstream of the first solenoid valve 204.

The NO/N₂/O₂ mixture thus obtained is then, as previously, conveyed through the common backup line 140 which opens out at a backup outlet 141, i.e. a secondary outlet, in order to supply the backup gaseous mixture formed by NO/N₂/O₂, and then to be able to convey it, via a flexible conduit or the like, to a manual ventilation bag in order to carry out manual ventilation of the patient with the gaseous NO/N₂/O₂ mixture at the desired dose.

On the other hand, in the case of a serious failure of the device 1 with an electrical supply fault, no activation signal is generated by the actuation means 195 and/or used/processed by the piloting means 900, and/or the display screen cannot operate; therefore, in the case of activation of the backup mode via the actuation means 195, the device 1 goes back to entirely pneumatic backup mode, as explained with reference FIG. 2.

The NO supply device 1 according to the invention has a higher level of safety, by making it possible to adjust the NO content more precisely in the case of a minor defect or fault, without loss of electrical supply, or when the user wishes to change over to manual ventilation of the patient by BVM.

In general, the NO supply device 1 according to the invention is particularly suitable for use within an installation 100 for administration of therapeutic gas, based on NO (< 100 ppmv), O₂ (>21% by volume) and nitrogen, to a patient P, such as the installation 100 in FIG. 1, used for treating one or more patients suffering from a disease or medical condition giving rise to acute pulmonary hypertension, in particular pulmonary vasoconstrictions in adults or children, including the newborn, for example for treating PPHN, or pulmonary hypertension in a person undergoing cardiac surgery.

In other words, according to another aspect, the invention thus also relates to a method for treating a person, called a patient, suffering from a disease or a medical condition that causes acute pulmonary hypertension, in particular pulmonary vasoconstriction, in adults or children, including neonates, for example for treating persistent pulmonary hypertension of the newborn (PPHN) in a neonate or baby or similar, or pulmonary hypertension in a person undergoing heart surgery, in which there is administered by inhalation, to said patient requiring it, a gaseous mixture containing oxygen (preferably >21% by volume), nitrogen and NO (preferably <100 ppmv), said gaseous mixture being supplied by an installation 100 according to the invention which administers therapeutic gas and comprises an NO supply device according to the invention, which NO supply device 1 is fed with an NO/N₂ mixture by at least one pressurized gas container 5, preferably several pressurized gas containers 5 such as NO/N₂ cylinders, and with oxygen by at least one pressurized oxygen container 52, such as an O₂ cylinder, and which NO supply device 1 is able and designed to supply: either an NO/N₂ mixture, via the main outlet orifice 210, to the respiratory gas circuit 3 connected to a medical ventilator 2 of the installation 100 for administration of therapeutic gas, or an NO/N₂/O₂ mixture, via the secondary orifice 141, to a manual insufflator, in particular a manual insufflation bag or bag valve mask (BVM).

Generally, within the context of the present invention, all of the terms “means” are considered to be wholly equivalent and can be substituted by the terms “device”; for example the terms “piloting means” can be replaced by “piloting device”, the terms “measurement means” can be replaced by “measurement device”, etc.

1. NO supply device comprising:
piloting means supplied with electrical energy by electrical energy supply means,
a main gas circuit comprising at least one main NO line fluidically connecting at least one NO inlet port to a main outlet orifice in order to convey an NO/N₂ mixture from said at least one NO inlet port to said main outlet orifice, said main NO line comprising NO/N₂ flowrate control means controlledby the piloting means,
a backup circuit comprising a backup NO circuit and a backup O₂ circuit, in which:
a) the backup NO circuit comprises a backup NO line in fluidic communication with the main NO line, the backup NO line comprising a pneumatic valve making it possible to control the circulation of the NO/N₂ mixture in the backup NO line and a second solenoid valve normally in an open position, said second solenoid valve being controlled by the piloting means, and
b) the backup O₂ circuit comprises a main O₂ line in fluidic communication with an O₂ inlet port, said main O₂ line comprising:
a first control valve controlled by an actuation means that can be actuated by a user in order to control the circulation of the flow of oxygen in said main O₂ line, said actuation means being further configured to supply an activation signal to the piloting means in response to an actuation of said actuation means (195)-by the user, and
a second, pneumatic control valve arranged downstream of the first control valve,
and in which the piloting means comprise at least one microprocessor and are configured, in response to the receipt of the activation signal delivered after actuation of the actuation means by the user:
to command the second solenoid valve of the backup NO circuit to interrupt all circulation of NO/N₂ mixture through said second solenoid valve,
to control the NO/N₂ flowrate control means in such a way as to regulate the flowrate of the NO/N₂ mixture in the main gas circuit, and
to command a first solenoid valve, arranged on the main NO line downstream of the NO/N₂ flowrate control means and on the backup NO line of the backup NO circuit downstream of the pneumatic valve and the second solenoid valve, in such a way as to direct the flow of NO/N₂ mixture coming from the NO/N₂ flowrate control means, in a downstream portion of the backup NO line connecting to the main O₂ line in order to form a common backup line (140) in fluidic communication with a backup outlet.

2. The device according to claim 1, further comrpising:
oxygen flowrate measurement means arranged downstream of the first control valve and configured to supply at least one oxygen flowrate measurement to the piloting means, and
O₂ flowrate regulating mean arranged downstream of the oxygen flowrate measurement means.

3. The device according to claim 2, wherein:
it comprises NO content regulating means which are configured to allow a user to choose a desired NO content and to supply said desired NO content to the piloting means, and
the piloting means are configured to calculate a flowrate of NO/N₂ mixture to be supplied, corresponding to the desired NO content, and to operate the NO/N₂ flowrate control means in order to supply said calculated flowrate of NO/N₂ mixture.

4. The device according to claim 3, wherein the piloting means are configured to calculate the flowrate of NO/N₂ mixture to be supplied on the basis of the desired NO content, the oxygen flowrate measured by the oxygen flowrate measurement means and an NO content in the NO/N₂ mixture conveyed through the main NO line.

5. The device according to claim 3, further comprising a touch-controlled digital display screen, and the NO content regulating means comprise at least one touch-activated selection key displayed on the display screen .

6. The device according to claim 5, wherein said at least one touch-activated selection key is configured to allow the user to set or select a desired NO value of between 1 and 80 ppmv.

7. The device according to claim 4, wherein the NO content in the NO/N₂ mixture conveyed through the main NO line is between 100 and 1000 ppmv.

8. The device according to claim 1, wherein the main O₂ line is configured to cooperate pneumatically with the pneumatic valve in order to allow the passage of the NO/N₂ mixture into the backup NO line, after actuation of the actuation means by the user.

9. The device according to claim 1, wherein the piloting means are further configured, in the absence of actuation of the actuation means by the user, to ensure a normal functioning of the device by piloting the NO/N₂ flowrate control means in such a way as to convey the NO/N₂ mixture through the main gas circuit between said at least one NO inlet port and the main outlet orifice.

10. The device according to claim 1, wherein the first solenoid valve comprises several ways, .

11. The device according to claim 1, wherein the backup NO line of the backup NO circuit and the main O₂ line of the backup O₂ circuit are connected fluidically to each other at a junction site situated downstream of the O₂ flowrate control means of the main O₂ line and downstream of the second solenoid valve of the backup NO line.

12. The device according to claim 1, wherein the backup outlet is configured to be connected fluidically to a manual insufflator .

13. NO supply device comprising:
piloting means comprising at least one microprocessor,
a main gas circuit comprising at least one main NO line fluidically connecting at least one NO inlet port to a main outlet orifice in order to convey an NO/N₂ mixture from said at least one NO inlet port to said main outlet orifice, said main NO line comprising NO/N₂ flowrate control means controlled by the piloting means, and a first solenoid valve controlled by the piloting means and arranged on the main NO line downstream of the NO/N₂ flowrate control means,
a backup circuit comprising a backup NO circuit and a backup O₂ circuit, in which:
i. the backup NO circuit comprises a backup NO line in fluidic communication with the main NO line, the backup NO line comprising a pneumatic valve making it possible to control the circulation of the NO/N₂ mixture in the backup NO line and a second solenoid valve controlled by the piloting means, the first solenoid valve being further arranged on the backup NO line downstream of the pneumatic valve,
ii. the backup O₂ circuit comprises a main O₂ line in fluidic communication with an O₂ inlet port, said main O₂ line comprising:
a first control valve controlled by an actuation means that can be actuated by a user in order to control the circulation of the flow of oxygen in said main O₂ line, said actuation means being further configured to supply an activation signal to the piloting means in response to an actuation of said actuation means by the user,
a second, pneumatic control valve arranged downstream of the first control valve,
oxygen flowrate measurement means arranged downstream of the second, pneumatic control valve and configured to supply at least one oxygen flowrate measurement to the piloting means, and
O₂ flowrate regulating means arranged downstream of the oxygen flowrate measurement means,
and NO content regulating means which are configured to allow the user to choose a desired NO content and to supply said desired NO content to the piloting means, and
in which the piloting means are configured, in response to the receipt of the activation signal resulting from an actuation of the actuation means by the user:
to calculate a flowrate of NO/N₂ mixture to be supplied corresponding to the desired NO content chosen by the user,
to operate the NO/N₂ flowrate control means in order to supply the calculated flowrate of NO/N₂ mixture, and
to act on the first solenoid valve in order to allow the passage of the NO/N₂ flow from the downstream conduit portion of the main gas circuit, situated downstream of the NO/N₂ flowrate control means, to a downstream part of the backup NO line situated downstream of the first solenoid valve and connecting fluidically to the main O₂ line to form a common backup line in fluidic communication with a backup outlet.

14. NO supply device comprising:
piloting means,
a main gas circuit comprising at least one main NO line fluidically connecting at least one NO inlet port to a main outlet orifice in order to convey an NO/N₂ mixture from said at least one NO inlet port to said main outlet orifice, said main NO line comprising NO/N₂ flowrate control means controlled by the piloting means,
a backup circuit comprising a backup NO circuit and a backup O₂ circuit, in which:
i. the backup NO circuit comprises a backup NO line in fluidic communication with the main NO line, the backup NO line comprising a pneumatic valve making it possible to control the circulation of the NO/N₂ mixture in the backup NO line and a second solenoid valve controlled by the piloting means, and
ii. the backup O₂ circuit comprises a main O₂ line in fluidic communication with an O₂ inlet port, said main O₂ line comprising:
a first control valve controlled by an actuation means that can be actuated by a user in order to control the circulation of the flow of oxygen in said main O₂ line, said actuation means being further configured to supply an activation signal to the piloting means in response to an actuation of said actuation means by the user,
a second, pneumatic control valve arranged downstream of the first control valve,
oxygen flowrate measurement means arranged downstream of the second, pneumatic control valve and configured to supply at least one oxygen flowrate measurement to the piloting means, and
O₂ flowrate regulating means arranged downstream of the oxygen flowrate measurement means
and in which:
the main O₂ line is configured to cooperate pneumatically with the pneumatic valve by way of a conduit section fluidically connecting the main O₂ line, downstream of the second control valve, to said pneumatic valve in order to the passage of the NO/N₂ mixture into the backup NO line in response to an actuation of the actuation means by the user,
the backup NO line of the backup NO circuit is connected fluidically to the main NO line downstream of the NO/N₂ flowrate control means, by way of a first solenoid valve (204), controlled by the piloting means, arranged on the main NO line downstream of the NO/N₂ flowrate control means and on the backup NO line of the backup NO circuit downstream of the pneumatic valve, said first solenoid valve (204) being configured to direct the flow of NO/N₂ mixture:
either in a downstream portion of the main NO line comprising the main outlet orifice,
or in a downstream portion of the backup NO line connecting to the main O₂ line, and
the backup NO line of the backup NO circuit and the main O₂ line of the backup O₂ circuit are connected fluidically to each other in order to form a common backup line in fluidic communication with a backup outlet.

15. Installationfor administering NO-containing therapeutic gas to a patient, comprising an NO supply device according to claim 1 which is fed with NO/N₂ mixture by at least one pressurized gas container and with oxygen by a pressurized oxygen container, said NO supply device supplying:
either an NO/N₂ mixture via the main outlet orifice, to a respiratory gas source connected to a medical ventilator,
or an NO/N₂/O₂ mixture via the backup orifice, to a manual insufflator or bag valve mask.

16. The device according to claim 1, wherein:
it comprises NO content regulating means which are configured to allow a user to choose a desired NO content and to supply said desired NO content to the piloting means, and
the piloting means are configured to calculate a flowrate of NO/N₂ mixture to be supplied, corresponding to the desired NO content, and to operate the NO/N₂ flowrate control means in order to supply said calculated flowrate of NO/N₂ mixture.

17. Installation for administering NO-containing therapeutic gas to a patient, comprising an NO supply device according to claim 2 which is fed with NO/N₂ mixture by at least one pressurized gas container and with oxygen by a pressurized oxygen container, said NO supply device supplying:
either an NO/N₂ mixture via the main outlet orifice, to a respiratory gas source connected to a medical ventilator,
or an NO/N₂/O₂ mixture via the backup orifice, to a manual insufflator or bag valve mask.

18. Installation for administering NO-containing therapeutic gas to a patient, comprising an NO supply device according to claim 3 which is fed with NO/N₂ mixture by at least one pressurized gas container and with oxygen by a pressurized oxygen container, said NO supply device supplying:
either an NO/N₂ mixture via the main outlet orifice, to a respiratory gas source connected to a medical ventilator,
or an NO/N₂/O₂ mixture via the backup orifice, to a manual insufflator or bag valve mask.

19. Installation for administering NO-containing therapeutic gas to a patient, comprising an NO supply device according to claim 4 which is fed with NO/N₂ mixture by at least one pressurized gas container and with oxygen by a pressurized oxygen container, said NO supply device supplying:
either an NO/N₂ mixture via the main outlet orifice, to a respiratory gas source connected to a medical ventilator,
or an NO/N₂/O₂ mixture via the backup orifice, to a manual insufflator or bag valve mask.

20. Installation for administering NO-containing therapeutic gas to a patient, comprising an NO supply device according to claim 5 which is fed with NO/N₂ mixture by at least one pressurized gas container and with oxygen by a pressurized oxygen container, said NO supply device supplying:
either an NO/N₂ mixture via the main outlet orifice, to a respiratory gas source connected to a medical ventilator,
or an NO/N₂/O₂ mixture via the backup orifice, to a manual insufflator or bag valve mask.
